(19) United States
(12) Patent Application Publication
(10) Pub. No.: US 2007/0276206 A1
(43) Pub. Date: Nov. 29, 2007

(54) Skin Evaluating Method And Skin Evaluation

(75) Inventors: Hideyo Takeuchi, Okazaki-shi (JP); Masahiro Hoguro, Kasugai-shi (JP); Tatsuki Yoshimine, Nagoya-shi (JP)

Correspondence Address: OLIFF & BERRIDGE, PLC, P.O. BOX 19928, ALEXANDRIA, VA, 22320, US

(73) Assignee: Kabushiki Kaisha DDS, Nagoya-shi (JP)

(21) Appl. No.: 10/586,968

(22) PCT Filed: Feb. 28, 2005
(86) PCT No.: PCT/JP05/03315

§ 371 Date: Nov. 13, 2006

(30) Foreign Application Priority Data
Dec. 27, 2004 (JP) 2004-375392

(51) Int. Cl. A61B 5/00

(52) U.S. Cl. 600/306

(57) ABSTRACT

A part of the image input from a fingerprint sensor (S1) is cut out (S3), and a fundamental frequency is calculated by using second-order linear predictive analysis (S5). A total value S of the fundamental frequencies in a direction x and a direction y is calculated so as to be used as a grading scale of fineness in texture (S7). A prepared threshold is compared with the grading scale S obtained at S7, and the fineness in texture of a skin condition is evaluated in three grades (S9 to S17). Further, a ratio of the fundamental frequency of the subject image in the direction x to the fundamental frequency in the direction y is calculated so as to be used as a grading scale relating to texture running (S19). Similar grading scales are obtained by rotating the image, and the texture running is determined into two grades based on the grading scales (S31 to S35). Results of determining the fineness in texture and the texture running are displayed on a display screen (S37).

### TECHNICAL FIELD

The present invention relates to a skin evaluating method and a skin evaluating device.

### BACKGROUND ART

In general, a lot of people hope to have healthy and beautiful skin. In order to fulfill a people's desire to be beautiful, many companies have developed and sold wide variety of cosmetics and beauty appliances. Since cosmetics and beauty appliances are compatible with some people but are not compatible with the other people, it is necessary to select a beauty regimen suitable for each skin type of different people and skin conditions at that time in order to exert a sufficient effect of the cosmetics and beauty appliances. It is, therefore, very important to diagnose a skin condition.

The skin (occasionally called as cutis) condition is generally evaluated based on “texture”. The texture means net-like contour which spreads on the surface of the skin, and it is composed of “skin hillock” which is a high portion, “skin groove” between the skin hillocks, and “hair pouch”. The “texture of skin” is generally evaluated by “fineness in texture” and “texture shape”. “Fine skin” means a condition that uniform and beautiful skin relief continues. Further, a condition that the skin has stripes is a condition that “texture runs” (called also “fibrous skin”). A condition that the texture is well-shaped and does not have stripes is a condition that the textures shape is satisfactory.

Decade ago, the skin conditions were diagnosed by only specialists in cosmetic companies or the like. However, in the diagnosis of the skin conditions carried out by the specialists, an observer's intuitive determination is an important element. Consequently, the evaluation requires long-period proficiency, and thus not everyone can make such an evaluation readily. In order to solve this problem, a skin diagnosing device which diagnoses a skin automatically is devised in recent years.

For example, Patent Document 1 proposes a skin diagnosing method whose grading scale is non-uniformity of a skin including procedures of: (1) obtaining a RGB image (color image) of a skin, (2) converting RGB into Lab image, (3) calculating a variation coefficient (dispersion) of an area or a position with respect to the entire image of a low luminance area and (4) evaluating non-uniformity of a skin using the variation coefficient.

Further, Patent Document 2 discloses a method of measuring the number of intersection points per unit area or an average area per intersection point when a point at which three or more skin grooves meet on the skin surface is one intersection point, and measuring roughness of the texture of the skin.

Patent Document 1: Japanese Patent No. 3351958

Patent Document 2: Japanese Patent Application Laid-Open No. 2001-170028

### DISCLOSURE OF THE INVENTION

### Problems to be Solved by the Invention

In the Patent Document 1, however, it is assumed that a skin image to be analyzed has comparatively good quality, and so a comparatively expensive camera which can obtain a color image needs to be used. And a threshold to evaluate a low luminance area in the Lab image is necessary. There is a problem that if a comparatively expensive camera with less individual difference is not used, it is difficult to set the threshold value.

In the Patent Document 2, binarization and thinning of lines are required. It is supposed that a camera with less individual difference which can set a binarization threshold or a skin image is obtained under a constant condition, and thus there is a problem that it is difficult to analyze an image with low quality obtained by an inexpensive camera.

In order to solve the above problems, the present invention is devised, and its object is to provide a skin evaluating method and a skin evaluating device which can evaluate a skin condition even in the case where an obtained image is unclear to a certain extent or in the case where image obtaining devices whose individual differences are large is used.

### Means to Solve the Problems

In order to achieve the above object, a skin evaluating method of the present invention analyzes a frequency of an input skin image and a skin condition is determined based on a frequency feature of the skin image obtained by the frequency analysis.

Further, according to the skin evaluating method of the present invention, the fundamental frequency of the skin image is extracted as the frequency feature by the frequency analysis, and when the fundamental frequency exceeds a predetermined threshold, the condition of the skin may be determined as satisfactory. The fundamental frequency in the present invention includes fundamental frequencies obtained by well-known frequency analysis and also by a zero cross method or the like which is a simple fundamental frequency calculating method.

According to the skin evaluating method of the present invention, when the frequency analysis of the skin image is performed in a direction X and a direction Y, respectively, the fundamental frequencies of the skin image in the direction X and the direction Y are extracted, a ratio of the fundamental frequency in the direction X to the fundamental frequency in the direction Y is calculated, and when the ratio is within a range of the predetermined threshold, the condition of the skin may be determined as satisfactory.

According to the skin evaluating method of the present invention, a second-order linear predictive analysis may be used as the frequency analysis.

According to the skin evaluating method of the present invention, the skin image may be input by a fingerprint sensor.

Further, a skin evaluating device of the present invention comprises: image input means for inputting a skin image; frequency analyzing means for analyzing a frequency of the skin image input by the image input means; feature extracting means for extracting a frequency feature of the skin image obtained by frequency analysis by means of the frequency analyzing means; and determining means for determining a condition of the skin based on the frequency feature extracted by the feature extracting means.

According to the skin evaluating device of the present invention, the feature extracting means extracts a fundamental frequency of the skin image as the frequency feature, and when the fundamental frequency exceeds a predetermined threshold, the determining means may determine that the condition of the skin is satisfactory.

Further, according to the skin evaluating device of the present invention, the frequency analyzing means analyzes the frequencies of the skin image in the direction X and the direction Y, respectively, the feature extracting means extracts the fundamental frequencies in the direction X and the direction Y of the skin image, and frequency ratio calculating means for calculating a ratio of the fundamental frequency in the direction X to the fundamental frequency in the direction Y extracted by the feature extracting means is provided, and when the ratio calculated by the frequency ratio calculating means is within a range of the predetermined threshold, the determining means may determine that the condition of the skin is satisfactory.

According to the skin evaluating device of the present invention, the frequency analyzing means may use a secondary linear predictive analysis.

According to the skin evaluating device of the present invention, the image input means may be composed of a fingerprint sensor.

### EFFECT OF THE INVENTION

According to the skin evaluating method of the present invention, the frequency of the input skin image is analyzed, and the condition of the skin is determined based on the frequency feature of the skin image obtained by analyzing the frequency. Such frequency feature can be obtained even when the input skin image is not clear. Therefore, the condition of the skin can be evaluated without depending on the quality of the input skin image.

According to the skin evaluating method of the present invention, the fundamental frequency may be extracted as the frequency feature. A correlation that when the fundamental frequency is high, the condition of the skin is satisfactory, namely, the texture is fine is confirmed, so that the condition of the skin can be quantized and determined by the fundamental frequency.

According to the skin evaluating method of the present invention, when the ratio of the fundamental frequency in the direction X to the fundamental frequency in the direction Y is obtained, the texture running of the skin can be calculated, and the condition of the skin can be evaluated by the degree of the running based on the texture shape. Particularly, by elevating the texture running as well as the fineness in texture, the skin can be evaluated more precisely.

According to the skin evaluating method of the present invention, using the second-order linear predictive analysis as the frequency analysis, the skin can be evaluated by a technique which is well known in sound and fingerprint authorization fields.

According to the skin evaluating method of the present invention, the skin image may be input by using the fingerprint sensor, and in this case, a mechanism that makes an imaging distance between the skin and a camera lens constant, a labor for focusing and the like are not necessary unlike the case of imaging by means of a camera. As a result, the skin image to be evaluated can be easily obtained.

According to the skin evaluating device of the present invention, the frequency of the input skin image is analyzed, and the condition of the skin is determined based on the frequency feature of the skin image obtained by analyzing the frequency. Such frequency feature can be obtained even when the input skin image is not clear. Therefore, the condition of the skin can be evaluated without depending on the quality of the input skin image.

According to the skin evaluating device of the present invention, the fundamental frequency may be extracted as the frequency feature. A correlation that when the fundamental frequency is high, the condition of the skin is satisfactory, namely, the texture is fine is confirmed, so that the condition of the skin can be quantized and determined by the fundamental frequency.

According to the skin evaluating device of the present invention, when the ratio of the fundamental frequency in the direction X to the fundamental frequency in the direction Y is obtained, the texture running of the skin can be calculated, and the condition of the skin can be evaluated based on the degree of the running and the texture shape. Particularly, when the texture running as well as the fineness in texture is evaluated, the skin can be evaluated more precisely.

Further, according to the skin evaluating device of the present invention, when the second-order linear predictive analysis is used as the frequency analysis, the skin can be evaluated by a technique which is well known in sound and fingerprint authorization fields.

Further, according to the skin evaluating device of the present invention, the skin image may be input by using the fingerprint sensor, and in this case, a mechanism that makes an imaging distance between the skin and a camera lens constant, a labor for focusing and the like are not necessary unlike the case of imaging by means of a camera. As a result, the skin image to be evaluated can be easily obtained.

### BEST MODE FOR CARRYING OUT THE INVENTION

An embodiment to which the present invention is applied is explained below with reference to drawings. In the following embodiment, a program for executing a skin evaluating method of the present invention by means of a computer is mounted to a mobile telephone with fingerprint sensor, and the mobile telephone is operated as a skin evaluating device of the present invention. The outline of the embodiment is explained. The skin evaluating program incorporated into the mobile telephone is activated, an operator makes the fingerprint sensor mounted to the mobile telephone read a skin image, a skin condition of the input skin image is evaluated by the skin evaluating program, and a result is displayed on a display screen.

A constitution of the mobile telephone is explained with reference to FIGS. 1 and 2. FIG. 1 is an appearance view of the mobile telephone 1. FIG. 2 is a block diagram illustrating an electric constitution of the mobile telephone 1.

As shown in FIG. 1, the mobile telephone 1 is provided with a display screen 2, a ten key input section 3, a jog pointer 4, a phone call start button 5, a phone call end button 6, a microphone 7, a speaker 8, function selecting buttons 9 and 10, a fingerprint sensor 11 as image input means, and an antenna 12 (see FIG. 2). The ten key input section 3, the jog pointer 4, the phone call start button 5, the phone call end button 6 and the function selecting buttons 9 and 10 compose a key input section 38 (see FIG. 2).

The fingerprint sensor 11 may be a capacitance type sensor, an optical sensor, thermal type sensor, electric-field type sensor, flat type sensor or line type sensor as long as the sensor can obtain a part of or the entire part of an fingerprint image as fingerprint information. In this embodiment, the line type sensor is used, and the operator holds the mobile telephone 1 and runs the fingerprint sensor 11 along the skin so that a skin image is read by the fingerprint sensor 11.

As shown in FIG. 2, the mobile telephone 1 is provided with an analog front end 36 which amplifies a sound signal from the microphone 7 and a sound output from the speaker 8, a sound codec section 35 which converts the sound signal amplified by the analog front end 36 into a digital signal and converts a digital signal received from a modem section 34 into an analog signal so that the digital signal can be amplified by the analog front end 36, the modem section 34 which performs modulation and demodulation, and a transmitting/receiving section 33 which amplifies and detects an electric wave received from the antenna 12, and modulates and amplifies a carrier signal based on the signal received from the modem section 34.

Further, the mobile telephone 1 is provided with a control section 20 which control the entire mobile telephone 1, and the control section 20 contains a CPU 21, a RAM 22 which temporarily stores data therein and a clock function section 23. The RAM 22 is used as a work area in a process mentioned later, and storage areas, such as an area where skin contour obtained from the fingerprint sensor 11 which is converted into a density value is stored and an area where a calculated result in the process, mentioned later, is stored, are prepared. The control section 20 is connected to the key input section 38 which inputs characters or the like, the display screen 2, the fingerprint sensor 11, a nonvolatile memory 30, and a melody generator 32 which generates ring alert. The melody generator 32 is connected to the speaker 37 which produces the ring alert generated from the melody generator 32. The nonvolatile memory 30 is provided with an area where various programs to be executed by the CPU 21 of the control section 20 are stored, an area where various initial setting values are stored, an area where predetermined various threshold are stored, and the like.

Next, a skin evaluating process to be executed in the mobile telephone 1 having the above constitution is explained below with reference to FIGS. 3 to 8. FIG. 3 is a flowchart illustrating a flow of the skin evaluating process. FIG. 4 is an explanatory diagram illustrating a sample of a skin image input by the fingerprint sensor and a small area cut out from the skin image. FIG. 5 is a graph illustrating a fundamental frequency extracted from the skin image. FIG. 6 is an explanatory diagram illustrating samples of the skin image and the fundamental frequency. FIG. 7 is an explanatory diagram illustrating samples of the skin image and the fundamental frequency when a grading scale of texture running is obtained. FIG. 8 is an explanatory diagram illustrating a sample of a display screen showing results of determining skin evaluation.

As shown in FIG. 3, when the skin evaluating process is started, a skin image 100 shown in FIG. 4 input from the fingerprint sensor 11 is acquired (S1). As shown in FIG. 4, as to the size of the skin image acquired in this embodiment, the width is 224 pixels, and the height is arbitrary (H pixel). In order to select an evaluation subject from the acquired skin image, a small area 101 whose gradation value is the largest is cut out (S3). The size of the small area cut out here is 128 pixels×128 pixels. The size is not, however, limited to this. A fundamental frequency (skin pitch) of the cut-out small area is obtained (S5). The fundamental frequency is obtained according to the following method by using a second-order linear predictive analysis.

Each line of waveform Fi,j in a direction X of the small area image cut out at S3 is multiplied by the Hamming window as expressed by the following formula (1), so that Hi,j is obtained. At this point, i=0,1, . . . M−1, j=0,1, . . . N−1: M denotes the pixel in a direction y (128 in this embodiment), N is the pixel in a direction x (128 in this embodiment).

$$[ Formula 1 ] \\ {H}_{i , j} = [ 0.54 - 0.46 cos ( \frac{2 π}{128} j ) ] {F}_{i , j} \qquad ( 1 )$$

Autocorrelation coefficients ri,1 and ri,2 are obtained based on the obtained Hi,j according to the following formula (2).

$$[ Formula 2 ] \\ {r}_{i , 1} = \frac{{\sum }_{j = 0}^{N - 2} {H}_{i , j} {H}_{i , j + 1}}{{\sum }_{j = 0}^{N - 1} {H}_{i , j} {H}_{i , j}} \\ {r}_{i , 2} = \frac{{\sum }_{j = 0}^{N - 3} {H}_{i , j} {H}_{i , j} + 2}{{\sum }_{i = 0}^{N - 1} {H}_{i , j} {H}_{i , j}} \qquad ( 2 )$$

Linear predictive coefficients αi,0 and αi,1 are obtained based on the autocorrelation coefficients ri,1 and ri,2 according to the following formula (3).

$$[ Formula 3 ] \\ ( \begin{matrix}{α}_{i , 0} \\ {α}_{i , 1}\end{matrix} ) = \frac{1}{{r}_{i , 1}^{2} - 1} ( \begin{matrix}{r}_{i , 1} ( 1 - {r}_{i , 2} ) \\ {r}_{i , 2} - {r}_{i , l}^{2}\end{matrix} ) \qquad ( 3 )$$

Finally, a normalized resonance frequency fi which is normalized from 0 to π is calculated based on the linear predictive coefficients αi,0 and αi,1 according to the following formula (4).

$$[ Formula 4 ] \\ {f}_{i} = - {tan}^{- 1} \frac{\sqrt{4 {α}_{i , 1} - {α}_{i , 0}^{2}}}{{α}_{i , 0}} \qquad ( 4 )$$

Since the fundamental frequency fi in the direction x is calculated according to the formulas (2) to (4), similarly the linear predictive coefficients αj,0 and αj,1 are obtained based on the autocorrelation coefficients rj,1 and rj,2, and the normalized resonance frequency fj is calculated so that the fundamental frequency in the direction y is obtained. The fundamental frequencies obtained in such a manner is as shown in FIG. 5.

Although the fundamental frequency is obtained by the second-order linear predictive analysis, the autocorrelation coefficients obtained according to the formula (2) may be used directly so that the fundamental frequency is calculated. Further, the fundamental frequency may be calculated by using a zero cross method which is widely used as simple frequency analysis. In the case where the zero cross method is used, the fundamental frequency should be the zero cross number, which is calculated by counting the number that the pixel of a certain one line of a skin image crosses a threshold previously obtained in an experiment or a fixed value. When the zero cross number is large, the fundamental frequency is high.

The correlation exists that as the texture of the skin image is finer, the fundamental frequency obtained in the above manner becomes higher, and as the texture is coarser, the fundamental frequency becomes lower. When the total of the fundamental frequencies of 128 lines calculated in this embodiment is obtained, therefore, the total value can be used as the grading scale of the fineness in the texture because when the total value is larger, the texture is more fine and when the total value is smaller, the texture is more coarse. The total value S of the fundamental frequency fx (i) in the direction x and the fundamental frequency fy (i) in the y direction is calculated according to the following formula (5) (S7).

$$[ Formula 5 ] \\ S = ( {\sum }_{i = 0}^{127} fx ( i ) + {\sum }_{i = 0}^{127} fy ( i ) ) / 2 \qquad ( 5 )$$

In the formula (5), the grading scale is the total of the fundamental frequency in the direction x and the fundamental frequency in the direction y, but the grading scale may be calculated only by the fundamental frequency in the direction x or the fundamental frequency in the direction y.

Next, the fineness in the texture is evaluated by using the grading scale of the texture obtained at S7. In this embodiment, the evaluation of the fineness in the skin texture is classified into three grades, and two thresholds are prepared. For example, the first threshold is set to 60, and the second threshold is set to 40. When the grading scale of the texture is not less than the higher first threshold, the skin has the fine texture, namely, the skin condition is satisfactory, and when the grading scale is between the thresholds 1 and 2, the skin is in an intermediate condition, and when the grading scale is less than the second threshold, the texture is not much fine, namely, the skin requires skin care.

A determination is made as to whether the grading scale is not less than the first threshold (S9). When the texture grading scale is not less than the first threshold (YES at S9), the evaluation of the fineness in texture is determined as “satisfactory”, and the evaluation is temporarily stored in the RAM 22 (S11). The relationship between the skin image evaluated as “satisfactory” and the grading scale S of the texture is data 01, 02, 03 and 04 in FIG. 6, for example. In all the data, the texture grading scale S exceeds 60, and it is found that the texture is in order from the visual inspection of the skin image.

When the texture grading scale is not the first threshold or more (NO at S9), a determination is made as to whether the texture grading scale is the second threshold or more (S13). When the texture grading scale is the second threshold or more (YES at S13), the evaluation of the fineness in texture is determined as “intermediate”, and the evaluation is temporarily stored in the RAM 22 (S15). The relationship between the skin image evaluated as “intermediate” and the texture grading scale S is data 05, 06 and 07 in FIG. 6, for example. In all the data, the texture grading scale S is between 40 and 60. When the skin image is visually inspected, it cannot be said that the texture is in order but it cannot be said that the texture is rough.

When the texture grading scale is not the second threshold or more (NO at S13), the evaluation relating to the fineness in is determined as “defective”, and the evaluation is temporarily stored in the RAM 22 (S17). A relationship between the skin image evaluated as “defective” and the texture grading scale S is, for example, data 08, 09, 10, 11 and 12 in FIG. 6. In all the data, the texture grading scale S is less than 40, and it is found from the visual inspection of the skin image that the texture is rough. As observed above, there is seen a correlation between the skin condition and the grading scale S.

Next, a texture shape is evaluated. The evaluation of the texture shape is made by calculating a ratio of the fundamental frequency of the subject image obtained at S3 in the direction x to the fundamental frequency in the direction y. When the fundamental frequency in the direction y is higher than the fundamental frequency in the direction x, the texture runs laterally. For example, in the skin images 110 and 111 of FIG. 7, the value of the fundamental frequency Sy in the direction y is larger than the value of the fundamental frequency Sx in the direction x, and it can be observed from the image that the texture runs laterally. On the other hand, when the fundamental frequency Sy is lower than the fundamental frequency Sx in the direction x, the texture tends to run vertically. By using this property, the grading scale for determining the running of the texture is obtained according to the following formula (6) (S19).
Na=MAX(Sx,Sy)/MIN(Sx,Sy) (6)

A grading scale Na calculated by the formula (6) is the ratio of the fundamental frequency in the direction x to the fundamental frequency in the direction y. When N≈1, the texture is in order. When Na>>1, it can be determined that the texture runs.

In this method, however, when the running direction has a tilt which is close to 45°, the Sx and Sy obtains similar values, and thus it is difficult to detect the running. In order to avoid this problem, the ratio of pitches may be again obtained from an image obtained by rotating the cut-out image. The rotated image may be only a 45° rotated image, or for example, 22.5°, 45° and 67.5° rotated images in order to heighten the accuracy.

Therefore, after the grading scale of the running of the original image is calculated at S19, a determination is made as to whether a process for rotating the image in order to determine the running is ended (S21). The steps S23, S25 and S19 are repeated according to the number of the images to be rotated. When the image rotating process for determining the running is not yet ended (NO at S21), the subject skin image is rotated through a predetermined angle as shown in FIG. 7, so that a rotated image 112 is obtained (S23). Next, the fundamental frequencies in the direction x and the direction y are calculated from the rotated image 112 (S25). The image 113 in FIG. 7 shows the fundamental frequencies calculated from the rotated image.

Next, the sequence returns to S19, and the grading scale Na of the image rotated through this angle is obtained according to the formula (6). The determination is again made as to whether the scheduled processes on all the rotated images are ended (S21). The grading scales Na of all the rotated images are obtained (YES at S21), the grading scales are again calculated according to the following formula (7) based on the grading scale N₀ of the original image and the grading scales of all the rotated images (S29). For example, in the case that the rotated images are 22.5°, 45°, and 67.5°, the calculation is based on N₀, N_{22.5}, N₄₅ and N_{67.5}.
N=MAX(N₀,N_{22.5},N₄₅,N_{67.5}) (7)

In the formula (7), the maximum value of the grading scale Na obtained from the respective images is adopted as the grading scale N of the texture running, but the grading scale N may be calculated by obtaining an average value and a total value of the respective grading scales Na. When the maximum value is obtained from N₀, N_{22.5}, N₄₅ and N_{67.5}, intervals between the four points are interpolated by Gaussian window or the like, so that the calculating accuracy of N is improved. When the skin evaluation process is executed by a computer whose processing speed is high enough, the image is rotated at every 1°, for example, so that the calculating accuracy of N can be improved.

Next, the texture shape is evaluated by using the grading scale of the texture running obtained at S29. In this embodiment, the evaluation of the texture shape of the skin includes two-grade evaluation such that the texture is in order or the texture runs. Since the grading scale obtained at S29 is the ratio of the fundamental frequency in the direction x to the fundamental frequency in the direction y, a determination is made as to whether the grading scale is an approximate value of 1 (S31). When the grading scale is the approximate value of 1 (YES at S31), the evaluation of the texture shape is “in order”, and this evaluation is temporarily stored in the RAM 22 (S33). When the grading scale is not the approximate value of 1 (NO at S31), the evaluation of the texture shape is “running”, and this evaluation is temporarily stored in the RAM 22 (S35).

The fineness in texture and the texture shape are evaluated by the above processes, and since the evaluations are stored in the RAM 22, both the evaluated results are called, and a result display screen shown in FIG. 8 is displayed on the display screen 2 of the mobile telephone 1 (S37). FIG. 8 illustrates an example where the fineness in texture is intermediate and the texture shape is in order. Like this display screen, a process for performing a comprehensive evaluation where the evaluations of the fineness in texture and the texture shape are generalized may be added. In this embodiment, the comprehensive evaluation is displayed as three-stage evaluations including A (satisfactory), B (intermediate) and C (defective: care is required).

According to the mobile telephone 1 which functions as the skin evaluating device of this embodiment, the fundamental frequency of the image read by the fingerprint sensor 11 is calculated so that the fineness in texture is determined. Further, the ratio of the fundamental frequency of the skin image in the direction x to the fundamental frequency in the direction y is calculated so that the texture shape is determined. Since the fundamental frequency can be calculated even when the skin image is not clear, the skin condition can be evaluated simply without a device such as an expensive camera. Since a load on the process is comparatively light, even when the program is incorporated into a device similar to the mobile telephone whose CPU ability is not much high, the process can be executed at high speed suitably.

In the above embodiment, the CPU 21 which executes the process for calculating the fundamental frequency at S5 of the flowchart in FIG. 3 functions as frequency analyzing means of the present invention. Further, the CPU 21 which executes the process for calculating the texture grading scale at S7 in the flowchart of FIG. 3 functions as feature extracting means of the present invention. Further, the CPU 21, which executes the process for determining the fineness in texture at S9 and S13 in the flowchart of FIG. 3 and executes the process for determining the texture shape at S31, functions as determining means of the present invention. The CPU 21, which executes the process for calculating the grading scale of the running at S19 in the flowchart of FIG. 3, functions as frequency ratio calculating means of the present invention.

In the above embodiment, the program for allowing the computer to execute the skin evaluating method of the present invention is incorporated into the mobile telephone so that the mobile telephone functions as the skin evaluating device. The embodiment of the present invention, however, is not limited to the above constitution, and the program may be read as an application program of a personal computer so as to be executed. Further, the skin image is input not only by the fingerprint sensor, and it may be imaged by a camera.

### BRIEF DESCRIPTION OF THE DRAWINGS

FIG. 1 is an appearance view of a mobile telephone 1;

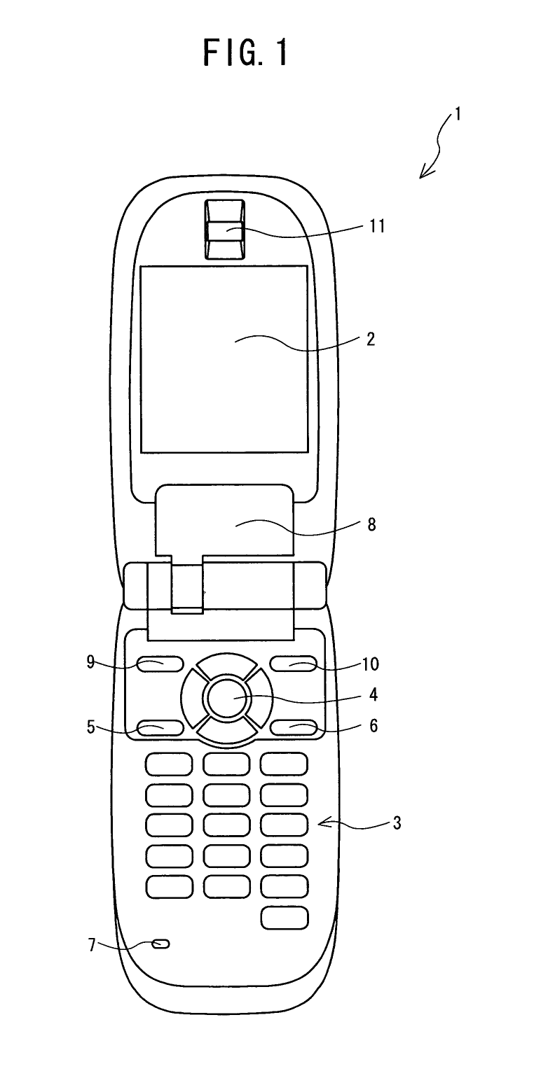

FIG. 2 is a block diagram illustrating an electric constitution of the mobile telephone 1;

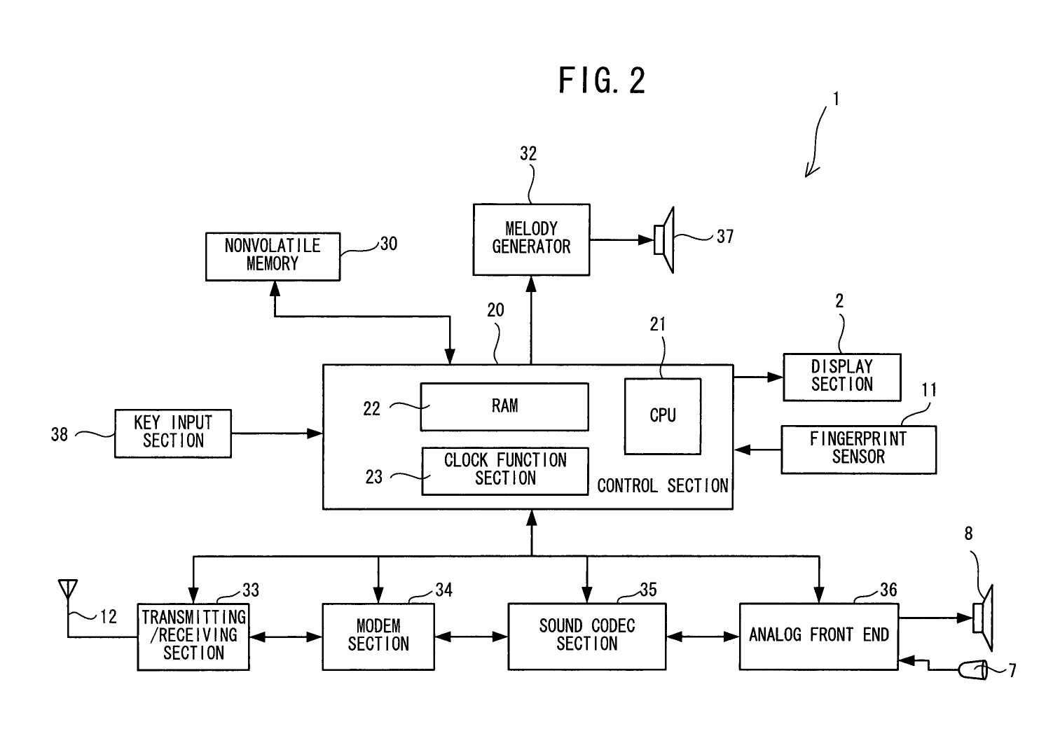

FIG. 3 is a flowchart illustrating a flow of a skin evaluating process;

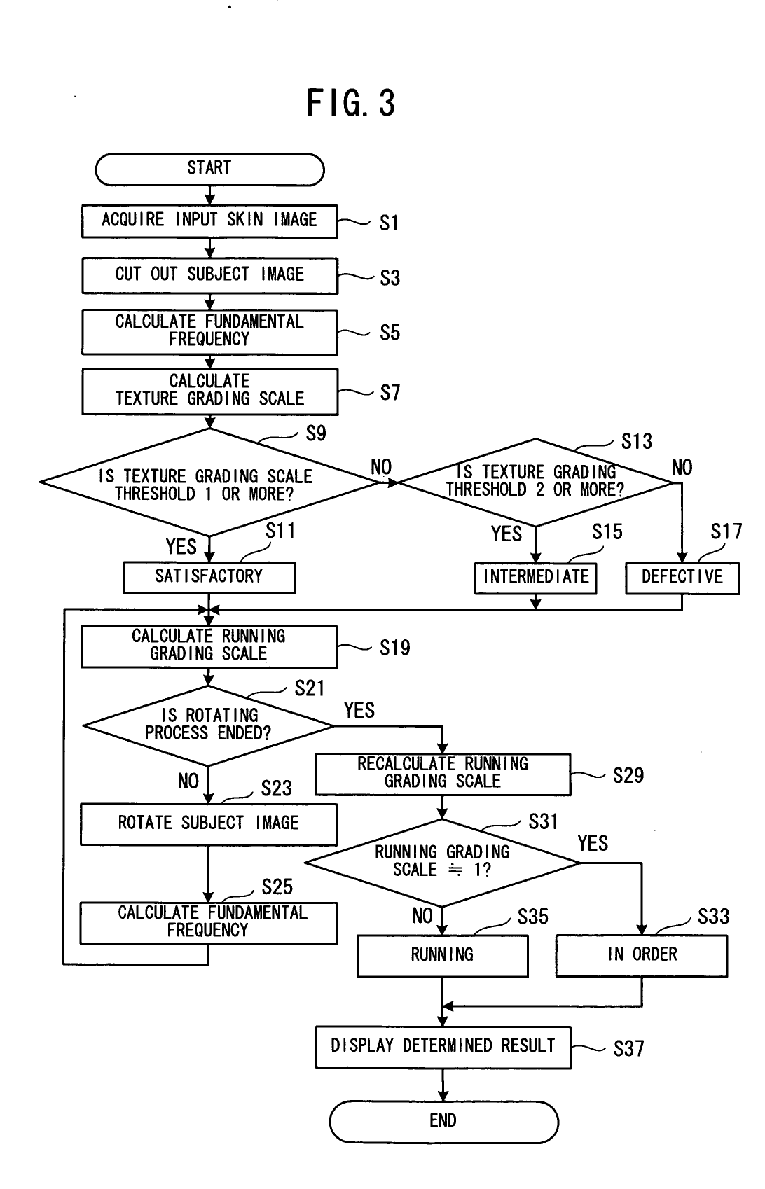

FIG. 4 is an explanatory diagram illustrating a sample of a skin image input by a fingerprint sensor and a small area cut out from the skin image;

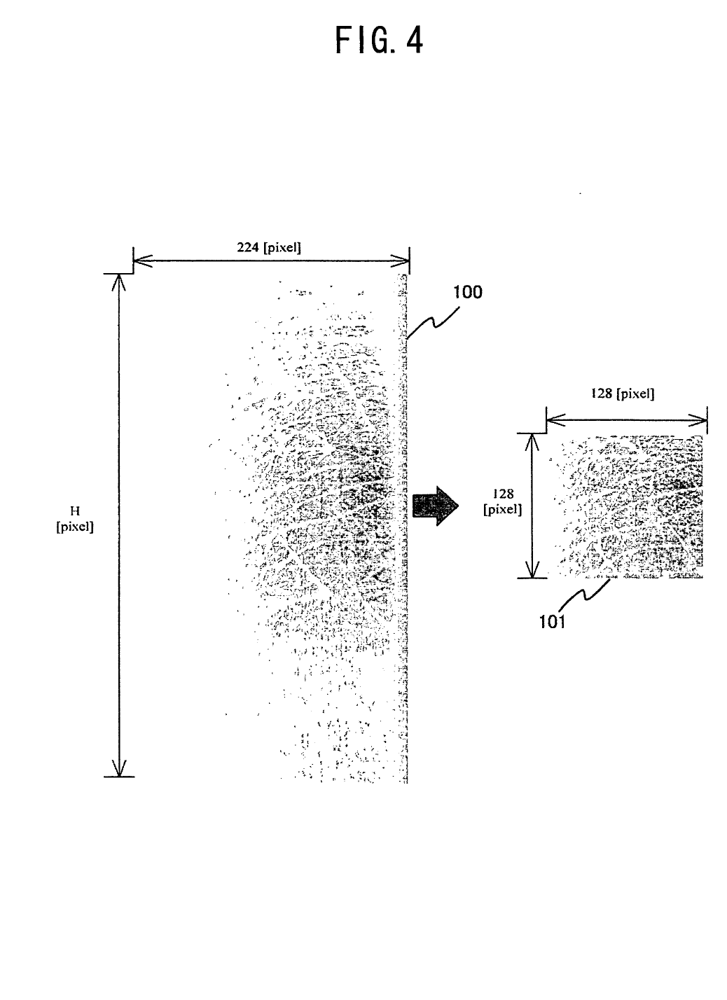

FIG. 5 is a graph illustrating a fundamental frequency extracted from the skin image;

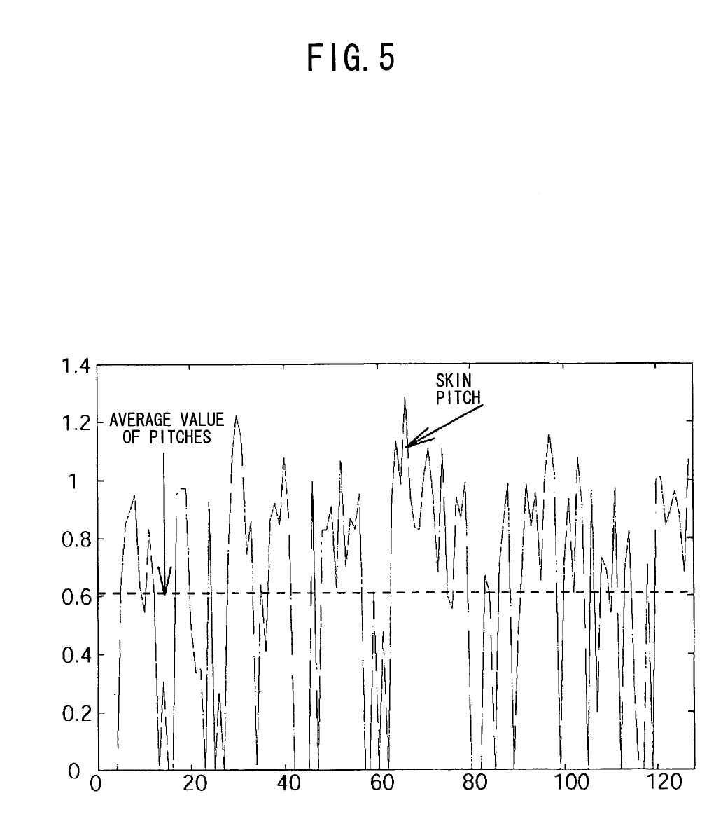

FIG. 6 is an explanatory diagram illustrating samples of the skin images and the fundamental frequencies;

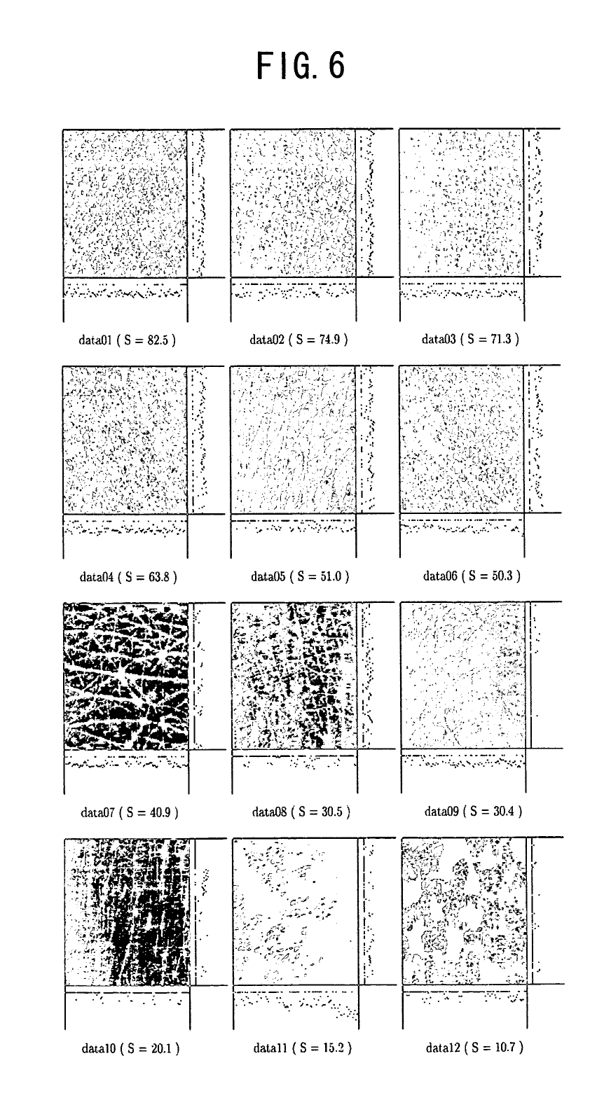

FIG. 7 is an explanatory diagram illustrating samples of the skin images and the fundamental frequencies when a grading scale of texture running is obtained; and

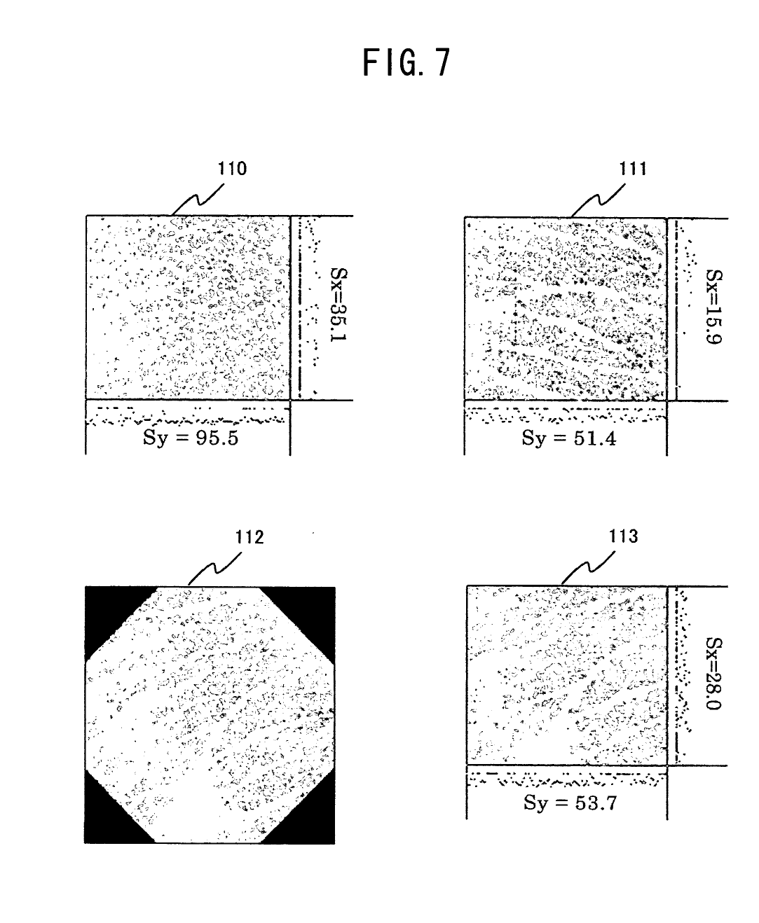

FIG. 8 is an explanatory diagram illustrating a sample of a display screen showing a determined result of the skin evaluation.

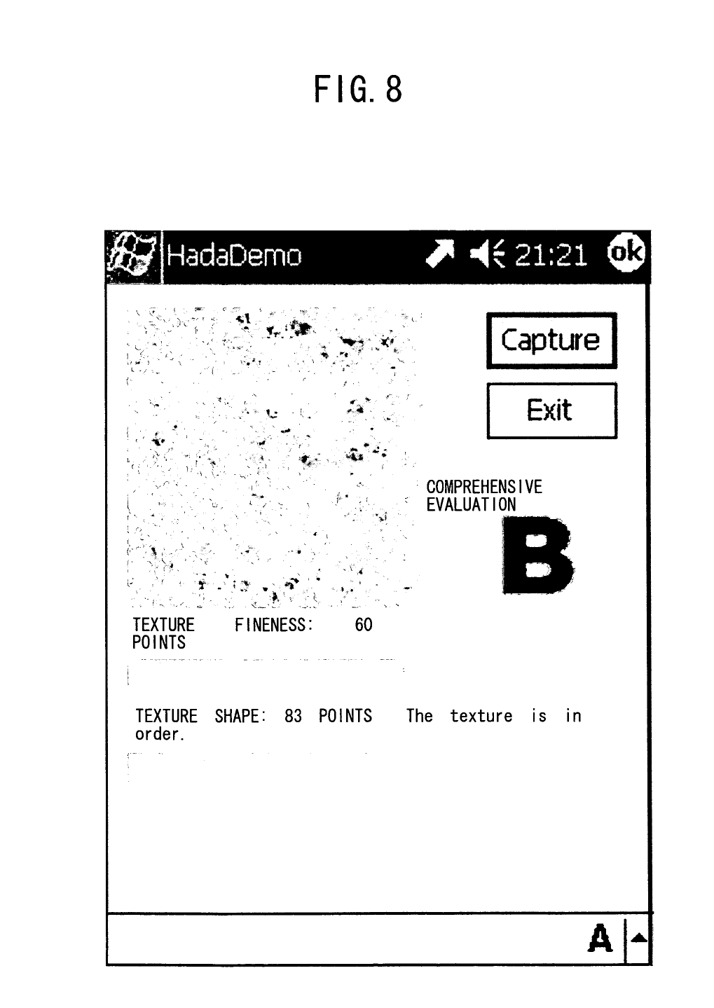

### EXPLANATION OF REFERENCE NUMERALS

1: mobile telephone
2: display screen
11: fingerprint sensor
21: CPU
22: RAM
20: control section
30: nonvolatile memory
100: skin image
101: small area

1-10. (canceled)

11. A skin evaluating method of analyzing a frequency of an input skin image and determining a condition of the skin based on a frequency feature of the skin image obtained by the frequency analysis.

12. The skin evaluating method according to claim 11, wherein a fundamental frequency of the skin image is extracted as the frequency feature by the frequency analysis, and when the fundamental frequency exceeds a predetermined threshold, the condition of the skin is determined as satisfactory.

13. The skin evaluating method according to claim 12, wherein the frequency analysis of the skin image is performed in a direction X and a direction Y, and the fundamental frequencies of the skin image in the direction X and the direction Y are extracted, a ratio of the fundamental frequency in the direction X to the fundamental frequency in the direction Y is calculated, and when the ratio is within a range of a predetermined threshold, the condition of the skin is determined as satisfactory.

14. The skin evaluating method according to claim 13, wherein second-order linear predictive analysis is used as the frequency analysis.

15. The skin evaluating method according to claim 12, wherein second-order linear predictive analysis is used as the frequency analysis.

16. The skin evaluating method according to claim 11, wherein the frequency analysis of the skin image is performed in a direction X and a direction Y, and the fundamental frequencies of the skin image in the direction X and the direction Y are extracted, a ratio of the fundamental frequency in the direction X to the fundamental frequency in the direction Y is calculated, and when the ratio is within a range of a predetermined threshold, the condition of the skin is determined as satisfactory.

17. The skin evaluating method according to claim 16, wherein second-order linear predictive analysis is used as the frequency analysis.

18. The skin evaluating method according to claim 11, wherein second-order linear predictive analysis is used as the frequency analysis.

19. The skin evaluating method according to claim 11, wherein the skin image is input by a fingerprint sensor.

20. A skin evaluating device comprising:
image input means for inputting a skin image;
frequency analyzing means for analyzing a frequency of the skin image input by the image input means;
feature extracting means for extracting a frequency feature of the skin image obtained by a frequency analysis by the frequency analyzing means; and
determining means for determining a condition of the skin based on the frequency feature extracted by the feature extracting means.

21. The skin evaluating device according to claim 20, wherein
the feature extracting means extracts a fundamental frequency of the skin image as the frequency feature,
when the fundamental frequency exceeds a predetermined threshold, the determining means determines that the condition of the skin is satisfactory.

22. The skin evaluating device according to claim 21, wherein
the frequency analyzing means analyzes the frequencies of the skin image in a direction X and a direction Y,
the feature extracting means extracts the fundamental frequencies in the direction X and the direction Y of the skin image; and further comprising frequency ratio calculating means for calculating a ratio of the fundamental frequency in the direction X to the fundamental frequency in the direction Y extracted by the feature extracting means; and
wherein the determining means determines that the condition of the skin is satisfactory when the ratio calculated by the frequency ratio calculating means is within a range of the predetermined threshold.

23. The skin evaluating device according to claim 22, wherein the frequency analyzing means uses second-order linear predictive analysis.

24. The skin evaluating device according to claim 21, wherein the frequency analyzing means uses second-order linear predictive analysis.

25. The skin evaluating device according to claim 20, wherein
the frequency analyzing means analyzes the frequencies of the skin image in a direction X and a direction Y,
the feature extracting means extracts the fundamental frequencies in the direction X and the direction Y of the skin image; and further comprising frequency ratio calculating means for calculating a ratio of the fundamental frequency in the direction X to the fundamental frequency in the direction Y extracted by the feature extracting means; and
wherein the determining means determines that the condition of the skin is satisfactory when the ratio calculated by the frequency ratio calculating means is within a range of the predetermined threshold.

26. The skin evaluating device according to claim 25, wherein the frequency analyzing means uses second-order linear predictive analysis.

27. The skin evaluating device according to claim 20, wherein the frequency analyzing means uses second-order linear predictive analysis.

28. The skin evaluating device according to claim 20, wherein the image input means is a fingerprint sensor.
